(19) United States
(12) Patent Application Publication
(10) Pub. No.: US 2023/0395200 A1
(43) Pub. Date: Dec. 7, 2023

(54) METHOD FOR PERFORMANCE EVALUATION OF A DETERGENT COMPOSITION

(71) Applicant: Novozymes A/S, Bagsvaerd (DK)

(72) Inventors: Liv Spaangner Christiansen, Gentofte (DK); Jun An, Beijing (CN)

(73) Assignee: Novozymes A/S, Bagsvaerd (DK)

(21) Appl. No.: 18/249,826

(22) PCT Filed: Oct. 18, 2021
(86) PCT No.: PCT/CN2021/124370

§ 371 Date: Apr. 20, 2023

(30) Foreign Application Priority Data
Oct. 20, 2020 (WO) PCT/CN2020/122138
Dec. 21, 2020 (WO) PCT/CN2020/137939

(51) Int. Cl. G16C 20/30; G16C 60/00

(52) U.S. Cl. CPC G16C 20/30; G16C 60/00

(57) ABSTRACT

A computer-implemented method for performance evaluation of a detergent composition is disclosed, the method comprising obtaining one or more wash conditions including a first wash condition; obtaining ingredient data comprising first ingredient data and second ingredient data, the first ingredient data associated with one or more first ingredients including a first primary ingredient and the second ingredient data associated with one or more second ingredients including a second primary ingredient; determining a first detergent composition based on the ingredient data; determining a first performance of the first detergent composition; and outputting a first performance representation of the first performance.

The present disclosure relates to methods and devices for performance evaluation of a detergent composition and/or optimization of a detergent composition.

### BACKGROUND

Detergent development may be a time-consuming, costly and cumbersome process not necessarily leading to an optimum detergent composition.

### SUMMARY

Accordingly, there is a need for devices, systems, and methods that simplifies and improves the detergent development process of a detergent composition.

A method, such as a computer-implemented method, is disclosed. The method may be a method for performance evaluation of a detergent composition. The method may be a method for optimization of a detergent composition. The method comprises obtaining, e.g. via an interface of an electronic device, one or more wash conditions including a first wash condition; obtaining, e.g. via the interface of the electronic device, ingredient data comprising first ingredient data and/or second ingredient data, the first ingredient data optionally associated with one or more first ingredients including a first primary ingredient and/or the second ingredient data optionally associated with one or more second ingredients including a second primary ingredient; and determining, e.g. with one or more processors, a first detergent composition, e.g. based on the ingredient data. The method optionally comprises determining, e.g. with one or more processors, a first performance of the first detergent composition; and outputting, e.g. via the interface of the electronic device, a first performance representation of the first performance and/or the first detergent composition.

Further, an electronic device is provided, the electronic device comprising a processor, a memory, and an interface, wherein the processor is configured to obtain, e.g. via the interface, one or more wash conditions including a first wash condition; obtain e.g. via the interface and/or from the memory, ingredient data comprising first ingredient data and/or second ingredient data, the first ingredient data optionally associated with one or more first ingredients including a first primary ingredient and/or the second ingredient data optionally associated with one or more second ingredients including a second primary ingredient; and determine a first detergent composition, e.g. based on the ingredient data. The processor is optionally configured to determine a first performance of the first detergent composition; and output, e.g. via the interface, a first performance representation of the first performance and/or an ingredient representation of the first detergent composition.

Also disclosed is a detergent application configured to perform any of the methods as described herein. The detergent application may be run on an electronic device. The detergent application may be an app, a web application, or an executable file.

It is an important advantage of the present disclosure, that a detergent designer easily and readily can obtain an overview of different performances of a detergent composition in turn reducing the design time and allowing optimization of different performances of a detergent composition in an easy and resource-efficient way.

The present disclosure allows a detergent designer to optimize different performances including stain removal performance, cost performance, sustainability performance and ecolabel performance to customer's requirements in a time-efficient manner saving both time and resources on trials and tests, in turn simplifying and improving the development cycle.

### BRIEF DESCRIPTION OF THE DRAWINGS

The above and other features and advantages of the present disclosure will become readily apparent to those skilled in the art by the following detailed description of example embodiments thereof with reference to the attached drawings, in which:

FIG. 1 is a diagram illustrating an example system comprising an example electronic device and a server device according to this disclosure,

FIG. 2 is a flow-chart illustrating an example method according to the present disclosure,

FIG. 3 is an example user interface according to this disclosure,

FIG. 4 is an example user interface according to this disclosure,

FIG. 5 is an example user interface according to this disclosure,

FIG. 6 is an example user interface according to this disclosure,

FIG. 7 is an example user interface according to this disclosure,

FIG. 8 is an example performance representation,

FIG. 9 is an example performance representation,

FIG. 10 is an example performance representation,

FIG. 11 is an example performance representation,

FIG. 12 is an example performance representation,

FIG. 13 is an example performance representation, and

FIG. 14 is an example performance representation.

### DETAILED DESCRIPTION

Various example embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The figures are schematic and simplified for clarity, and they merely show details which aid understanding the disclosure, while other details have been left out. Throughout, the same reference numerals are used for identical or corresponding parts.

The present disclosure provides a method, e.g. for performance evaluation of a detergent composition and/or for optimization of a detergent composition. The method is optionally a method for generating a detergent composition.

The method comprising obtaining one or more wash conditions including a first wash condition, also denoted WC_1, and/or a second wash condition also denoted WC_2. The one or more wash conditions may comprise a third wash condition also denoted WC_3 and/or a fourth wash condition also denoted WC_4. The one or more wash conditions may comprise a fifth wash condition also denoted WC_5 and/or a sixth wash condition also denoted WC_6.

In the method, obtaining one or more wash conditions may comprise obtaining one or more wash conditions via a first user interface on a display of the electronic device. In other words, the method may comprise displaying a first user interface on a display and obtaining one or more wash conditions via the first user interface, e.g. via one or more wash condition drop-down lists of the first user interface. Each wash condition drop-down list may comprise a set of candidate wash conditions selectable by the user.

The first wash condition may be one of a wash temperature, a geographical location, such as a country or a region, a user type, a water hardness, a detergent form, and a detergent type.

The second wash condition may be different from the first wash condition. The second wash condition may be one of a wash temperature, a geographical location, such as a country or a region, a user type, a water hardness, a detergent form, and a detergent type.

The third wash condition may be different from the second wash condition and/or the first wash condition. The third wash condition may be one of a wash temperature, a geographical location, such as a country or a region, a user type, a water hardness, a detergent form, and a detergent type.

The fourth wash condition may be different from the third wash condition, the second wash condition and/or the first wash condition. The fourth wash condition may be one of a wash temperature, a geographical location, such as a country or a region, a user type, a water hardness, a detergent form, and a detergent type.

In one or more exemplary methods/devices where a wash condition, such as the first wash condition or the second wash condition, is a geographical location, the wash condition, such as the first wash condition or the second wash condition, may be selected from a set of candidate geographical locations. The set of candidate geographical locations may comprise at least two candidate geographical locations, such as three, four, five, or more candidate geographical locations. The set of candidate geographical locations may include one or more of Europe, Asia, US, China, Germany.

In one or more exemplary methods/devices where a wash condition, such as the first wash condition or the second wash condition, is a temperature, the first wash condition or the second wash condition may be selected from a set of candidate temperatures. The set of candidate temperatures may comprise at least two candidate temperatures, such as three, four, five, or more candidate temperatures. The set of candidate temperatures may include one or more of 20 degrees, 30 degrees, 40 degrees, 60 degrees, and 90 degrees. In one or more exemplary methods/devices, the set of candidate temperatures may include one or more of cold, tempered, warm, and hot.

In one or more exemplary methods/devices where a wash condition, such as the second wash condition or the third wash condition, is a water hardness, the second wash condition or the third wash condition may be selected from a set of candidate water hardnesses. The set of candidate water hardnesses may comprise at least two candidate water hardnesses, such as three, four, five, or more candidate water hardnesses. The set of candidate water hardnesses may include one or more water hardness ranges or e.g. soft, medium, and hard. Thereby, a detergent composition may be evaluated based on water hardness, which is a parameter affecting the performance of a detergent in turn leading to improved and more accurate detergent composition evaluation.

In one or more exemplary methods/devices where a wash condition, such as the third wash condition or the fourth wash condition, is a detergent form, the third wash condition or the fourth wash condition may be selected from a set of candidate detergent forms. The set of candidate detergent forms may comprise at least two candidate detergent forms, such as three, four, five, or more candidate detergent forms. The set of candidate detergent forms may include one or more of liquid, powder, and pod. Thereby, a detergent composition may be evaluated based on detergent form, which is a parameter affecting the performance of a detergent in turn leading to improved and more accurate detergent composition evaluation.

In one or more exemplary methods/devices where a wash condition, such as the fourth wash condition or the fifth wash condition, is a detergent type, the fourth wash condition or the fifth wash condition may be selected from a set of candidate detergent types. A detergent type is optionally indicative of the intended type of clothes to be washed with the detergent. The set of candidate detergent types may comprise at least two candidate detergent types, such as three, four, five, or more candidate detergent types. The set of candidate detergent types may include one or more of white, colour, black, sport, and wool. Thereby, a detergent composition may be evaluated based on detergent type, which is a parameter affecting the performance of a detergent in turn leading to improved and more accurate detergent composition evaluation.

In one or more exemplary methods/devices where a wash condition, such as the fifth wash condition or the sixth wash condition, is a user type, the fifth wash condition or the sixth wash condition may be selected from a set of candidate user types. The set of candidate user types may comprise at least two candidate user types, such as three, four, five, or more candidate user types.

A user type may be indicative of a wash pattern, such as a wash frequency and/or a water consumption per wash. The set of candidate user types may include one or more of a first user type responsible for washing for more than 3 people (large family), a second user type responsible for washing for more than 2-3 people (small family), and a third user type responsible for washing for only 1 person (single). Thereby, a detergent composition may be evaluated based on user type, which is a parameter affecting the performance of a detergent in turn leading to improved and more accurate detergent composition evaluation.

In one or more exemplary methods/devices, a wash condition, such as the first wash condition, the second wash condition, the third wash condition, the fourth wash condition, or the sixth wash condition, is selected from a detergent dosage size or volume, a washing machine type, such as top-filled or side-filled, a wash water volume, a rinse volume, a number of rinsing steps, a wash program, and a ballast composition.

The method comprises obtaining ingredient data optionally comprising first ingredient data also denoted ID_1 and/or second ingredient data also denoted ID_2. The ingredient data may comprise third ingredient data also denoted ID_3 and/or fourth ingredient data also denoted ID_4.

The ingredient data may comprise one or more of identifier(s), amount(s), cost(s), purity parameter(s), impurity parameter(s), form parameter(s), size parameter(s), lower limit(s), and upper limit(s) of one or more ingredients.

The first ingredient data ID_1 are optionally associated with one or more first ingredients including a first primary ingredient also denoted ING_1_1 and/or a first secondary ingredient also denoted ING_1_2. The first ingredients also denoted ING_1_1, where i=1, . . . , N, N being the number of first ingredients, may be of a first ingredient type such as surfactant or enzyme. The first ingredients may comprise two, three or more first ingredients, i.e. N may be 2 or more, such as 3, 4, 5, 6, 7, 8, or more.

In other words, the first ingredient data may comprise first primary ingredient data also denoted ID_1_1 indicative of a first primary ingredient ING_1_1 of the first detergent composition and/or first secondary ingredient data also denoted ID_1_2 indicative of a first secondary ingredient ING_1_2 of the first detergent composition. The first ingredient data may comprise a first ingredient type identifier also denoted IT_ID_1, e.g. indicative of the type or group of ingredients comprised in the first ingredient data. The first ingredient data may comprise a first ingredient sub-type identifier, e.g. indicative of the sub-type or sub-group of ingredients comprised in the first ingredient data. The first ingredient data may comprise one or more of identifier(s), amount(s), cost(s), purity parameter(s), impurity parameter(s), form parameter(s), size parameter(s), lower limit(s), and upper limit(s) of one or more first ingredients, such as surfactants.

The first ingredients may be of a first type, e.g. represented by a first ingredient type identifier. The first type may be selected from surfactant, enzyme, builder/chelator, polymer, bleaching system, foam regulator, perfume, colorant, and other ingredients. The other ingredients type may comprise one or more of foam regulator(s), perfume, colorant(s), builder(s)/chelator(s), polymer(s), bleaching system(s), foam regulator(s), perfume, and colorant(s).

In one or more example methods, the first primary ingredient is a surfactant and/or the first ingredient data ID_1 comprises first primary ingredient data ID_1_1 of the first primary ingredient. The first primary ingredient data optionally includes one or more of, such as all of: one or more identifiers, an amount AM_1_1, a cost CO_1_1, a purity parameter PP_1_1, an impurity parameter IP_1_1, a form parameter FP_1_1, a size parameter SP_1_1, a lower limit LL_1_1, and an upper limit UL_1_1. The one or more identifiers may comprise a first primary ingredient identifier I_ID_1_1 indicative of the first primary ingredient and optionally a first primary ingredient type identifier IT_ID_1_1 indicative of type of the first primary ingredient ING_1_1.

In one or more example methods, the first secondary ingredient is a surfactant and/or the first ingredient data comprises first secondary ingredient data ING_1_2 of the first secondary ingredient. The first secondary ingredient data optionally includes one or more of, such as all of: one or more identifiers, an amount AM_1_2, a cost CO_1_2, a purity parameter PP_1_2, an impurity parameter IP_1_2, a form parameter FP_1_2, a size parameter SP_1_2, a lower limit LL_1_2, and an upper limit UL_1_2. The one or more identifiers may comprise a first secondary ingredient identifier I_ID_1_2 indicative of the first secondary ingredient and optionally a first secondary ingredient type identifier IT_ID_1_2 indicative of type of the first secondary ingredient.

In one or more exemplary methods/devices/system, the first ingredients are surfactants (i.e. IT_ID_1 is surfactant or indicative of surfactant), ING_1_1 is LAS, ING_1_2 is AEA/SLES, and ING_1_3 is AEO.

The second ingredient data ID_2 are optionally associated with one or more second ingredients including a second primary ingredient also denoted ING_2_1 and/or a second secondary ingredient also denoted ING_2_2. The second ingredients also denoted ING_2j, where j=1, . . . , M, M being the number of second ingredients, may be of a second ingredient type such as surfactant or enzyme. The second ingredients may comprise two, three, four or more first ingredients, i.e. M may be 2 or more, such as 3, 4, 5, 6, 7, 8, or more.

The second ingredient type may be different from the first ingredient type. In one or more exemplary methods/electronic devices, the first ingredient type is surfactant and the second ingredient type is enzyme. In other words, the second ingredient data may comprise second primary ingredient data also denoted ID_2_1 indicative of a second primary ingredient ING_2_1 of the first detergent composition and/or second secondary ingredient data also denoted ID_2_2 indicative of a second secondary ingredient ING_2_2 of the first detergent composition. The second ingredient data may comprise a second ingredient type identifier also denoted IT_ID_2, e.g. indicative of the type or group of ingredients comprised in the second ingredient data. The second ingredient data may comprise a second ingredient sub-type identifier, e.g. indicative of the sub-type or sub-group of ingredients comprised in the third ingredient data. The second ingredient data may comprise one or more of identifier(s), amount(s), cost(s), purity parameter(s), impurity parameter(s), form parameter(s), size parameter(s), lower limit(s), and upper limit(s) of one or more second ingredients, such as enzymes.

The second ingredients may be of a second type, e.g. represented by a second ingredient type identifier. The second type may be selected from surfactant, enzyme, builder/chelator, polymer, bleaching system, foam regulator, perfume, colorant, and other ingredients. The other ingredients type may comprise one or more of foam regulator(s), perfume, colorant(s), builder(s)/chelator(s), polymer(s), bleaching system(s), foam regulator(s), perfume, and colorant(s).

In one or more example methods, the second primary ingredient is an enzyme and/or the second ingredient data ID_2 comprises second primary ingredient data ID_2_1 of the second primary ingredient. The second primary ingredient data optionally includes one or more of, such as all of: one or more identifiers, an amount AM_2_1, a cost CO_2_1, a purity parameter PP_2_1, an impurity parameter IP_2_1, a form parameter FP_2_1, a size parameter SP_2_1, a lower limit LL_2_1, and an upper limit UL_2_1. The one or more identifiers may comprise a second primary ingredient identifier I_ID_2_1 indicative of the second primary ingredient and optionally a second primary ingredient type identifier IR_ID_2_1 indicative of type of the second primary ingredient ING_2_1.

In one or more example methods, the second secondary ingredient is an enzyme and/or the second ingredient data comprises second secondary ingredient data ING_2_2 of the second secondary ingredient. The second secondary ingredient data optionally includes one or more of, such as all of: one or more identifiers, an amount AM_2_2, a cost CO_2_2, a purity parameter PP_2_2, an impurity parameter IP_2_2, a form parameter FP_2_2, a size parameter SP_2_2, a lower limit LL_2_2, and an upper limit UL_2_2. The one or more identifiers may comprise a second secondary ingredient identifier I_ID_2_2 indicative of the second secondary ingredient and optionally a second secondary ingredient type identifier IT_ID_2_2 indicative of type of the second secondary ingredient.

In one or more exemplary methods/devices/system, the second ingredients are enzymes (i.e. IT_ID_2 is enzyme or indicative of enzyme), ING_2_1 is protease, ING_2_2 is amylase, ING_2_3 is lipase, and ING_2_4 is mannanase.

In one or more example methods, obtaining ingredient data comprises obtaining third ingredient data associated with one or more third ingredients including a third primary ingredient.

The third ingredient data ID_3 are optionally associated with one or more third ingredients including a third primary ingredient also denoted ING_3_1 and/or a third secondary ingredient also denoted ING_3_2. The third ingredients also denoted ING_3_k, where k=1, . . . , K, K being the number of third ingredients, may be of a third ingredient type such as surfactant, enzyme, others, hydrotope, builder, co-builder, polymer, or bleach system. The third ingredients may comprise two, three or more third ingredients, i.e. K may be 2 or more, such as 3, 4, 5, 6, 7, 8, or more.

The third ingredient type may be different from the first ingredient type and/or the second ingredient type. In one or more exemplary methods/electronic devices, the third ingredient type is others. Surfactants and/or enzymes may be separated into sub-types, e.g. where a specific sub-type is assigned an ingredient type. Thus, the third ingredient data may comprise third primary ingredient data also denoted ID_3_1 indicative of a third primary ingredient ING_3_1 of the first detergent composition and/or third secondary ingredient data also denoted ID_3_2 indicative of a third secondary ingredient ING_3_2 of the first detergent composition. The third ingredient data may comprise a third ingredient type identifier also denoted IT_ID_3, e.g. indicative of the type or group of ingredients comprised in the third ingredient data. The third ingredient data may comprise a third ingredient sub-type identifier, e.g. indicative of the sub-type or sub-group of ingredients comprised in the third ingredient data. The third ingredient data may comprise a third ingredient sub-type identifier, e.g. indicative of the sub-type or sub-group of ingredients comprised in the third ingredient data. The third ingredient data may comprise one or more of identifier(s), amount(s), cost(s), purity parameter(s), impurity parameter(s), form parameter(s), size parameter(s), lower limit(s), and upper limit(s) of one or more third ingredients, such as others.

The third ingredients may be of a third type, e.g. represented by a third ingredient type identifier. The third type may be selected from surfactant, enzyme, builder/chelator, polymer, bleaching system, foam regulator, perfume, colorant, and other ingredients. The other ingredients type may comprise one or more of foam regulator(s), perfume, colorant(s), builder(s)/chelator(s), polymer(s), bleaching system(s), foam regulator(s), perfume, and colorant(s).

The third primary ingredient may be soap, citrate, or perfume. The third secondary ingredient may be different from the third primary ingredient and may be soap, citrate or perfume. Thus, in one or more exemplary methods/devices/system, the third ingredients are others (i.e. IT_ID_3 is others or indicative of others), ING_3_1 is citrate, and ING_3_2 is soap.

In one or more example methods, the third primary ingredient data and/or the third secondary ingredient data respectively includes one or more of, such as all of: one or more identifiers, an amount, a cost, a purity parameter, an impurity parameter, a form parameter, a size parameter, a lower limit, and an upper limit, of respective third primary ingredient and/or third secondary ingredient. The third primary ingredient data ID_3_1 may comprise a third primary ingredient identifier indicative I_ID_3_1 of the third primary ingredient and optionally a third primary ingredient type identifier IT_ID_3_1 indicative of type of the third primary ingredient. The third secondary ingredient data ID_3_2 may comprise a third secondary ingredient identifier I_ID_3_2 indicative of the third secondary ingredient and optionally a third secondary ingredient type identifier IT_ID_3_2 indicative of type of the third secondary ingredient ING_3_2.

The method comprises determining a first detergent composition, e.g. based on the ingredient data and/or the one or more wash conditions, such as based one or more, e.g. all, of first ingredient data ID_1, second ingredient data ID_2, and third ingredient data. In one or more exemplary methods/electronic devices, the first detergent composition is based on fourth ingredient data ID_4. Determining the first detergent composition may comprise including ingredients, such as ingredient identifier, cost(s), and/or amounts of the ingredient data in the detergent composition. Determining the first detergent composition may comprise updating a base detergent composition based on the ingredient data. The base detergent composition may be a default detergent composition, e.g. defining first ingredients, second ingredients, and third ingredients and optionally their ingredient type. Determining the first detergent composition may comprise selecting the base detergent composition, e.g. based on one or more wash conditions and/or a reference detergent composition, such as a primary reference detergent composition of the primary reference detergent.

A detergent composition, such as the first detergent composition, may comprise name/ingredient identifier and amount of the ingredients in the detergent with the detergent composition. Accordingly, a detergent composition may comprise a list of ingredients/ingredient identifier and amounts of the respective ingredients in the list. A detergent composition may comprise cost(s) of the different ingredients of the detergent composition. Amounts of a detergent composition may be relative amounts, e.g. % by weight or % by volume, and/or absolute amounts, e.g. by weight and/or by volume. A detergent composition may comprise ingredient type identifiers and/or ingredient sub-type identifiers of the respective ingredients. For example, surfactants of a detergent composition may be associated to or have assigned an ingredient type identifier indicative of surfactant and/or an ingredient sub-type identifier indicative of surfactant type, such as anionic, cationic, nonionic, semipolar, zwitterionic, or bio-based. For example, enzymes of a detergent composition may be associated to or have assigned an ingredient type identifier indicative of enzyme.

The method may comprise determining one or more performances, such as a plurality of performances and/or at least three performances, of the first detergent composition.

The method optionally comprises determining a first performance also denoted P_1 of the first detergent composition. Determining a first performance P_1 may comprise determining one or more, such as a plurality of, first performance metrics also denoted PM_1_r, where r=1, . . . , R, R being the number of first performance metrics of the first detergent composition. In other words, the first performance may comprise one or more first performance metrics of the first detergent composition. The first performance, e.g. first performance metric(s), may be based on ingredients and/or ingredient amounts of the first detergent composition. The first performance, e.g. first performance metric(s), may be based on one or more wash conditions, e.g. WC_1 and/or WC_2.

The first performance may be a stain removal performance. In other words, first performance metric(s) of the first performance may be indicative of the first detergent composition's stain removal capabilities. For example, the first performance metric PM_1_1 may be indicative of stain removal capability for a first stain type, such as AISE, JB, JBS, or CHIPS. The first performance metric PM_1_1 may be based on performance metrics for a plurality of different stains also denoted PM_1_1_1, PM_1_1_2, . . . , such as from 3 to 25 different stains, e.g. as defined by AISE stain set, STIWA stain set, JB stain set, JBS stain set, or CHIPS stain set.

The first performance metric PM_1_1 may be indicative of stain removal capability of stains of the AISE stain set, e.g. based on one or more wash conditions, e.g. in accordance with the first wash condition being indicative of Europe. The first performance metric PM_1_1 or PM_1_2 may be indicative of stain removal capability of stains of the STIWA stain set, e.g. based on one or more wash conditions, e.g. in accordance with the first wash condition being indicative of Germany. The first performance metric PM_1_1 or PM_1_2 may be indicative of stain removal capability of stains of the JB stain set, e.g. based on one or more wash conditions, e.g. in accordance with the first wash condition being indicative of country/region where the JB stain set is used for evaluating performance. The first performance metric PM_1_1 or PM_1_2 may be indicative of stain removal capability of stains of the JB stain set, e.g. based on one or more wash conditions, e.g. in accordance with the first wash condition being indicative of country/region where the JB stain set is used for evaluating performance. The first performance metric PM_1_1 or PM_1_2 may be indicative of stain removal capability of stains of the JBS stain set, e.g. based on one or more wash conditions, e.g. in accordance with the first wash condition being indicative of country/region where the JBS stain set is used for evaluating performance. The first performance metric PM_1_1 or PM_1_2 may be indicative of stain removal capability of stains of the CHIPS stain set, e.g. based on one or more wash conditions, e.g. in accordance with the first wash condition being indicative of country/region where the CHIPS stain set is used for evaluating performance, such as China.

Another first performance metric PM_1_2 may be indicative of stain removal capability for a second stain type, such as proteins or stains of a different stain set than the stain set of PM_1_1 as described above. Thus, the first performance metric PM_1_2 may be based on performance metrics for a plurality of different stains also denoted PM_1_2_1, PM_1_2_2, . . . , such as from 3 to 20 different protein stains.

A first performance metric PM_1_3 may be indicative of stain removal capability for a third stain type, such as starches. Thus, the first performance metric PM_1_3 may be based on performance metrics for a plurality of different stains also denoted PM_1_3_1, PM_1_3_2, . . . , such as from 3 to 20 different starch stains.

A first performance metric PM_1_4 may be indicative of stain removal capability for a fourth stain type, such as natural fats and oils. Thus, the first performance metric PM_1_4 may be based on performance metrics for a plurality of different stains also denoted PM_1_4_1, PM_1_4_2, . . . , such as from 3 to 20 different stains of natural fats and oils.

A first performance metric PM_1_5 may be indicative of stain removal capability for a fifth stain type, such as food thickeners and stabilizers. Thus, the first performance metric PM_1_5 may be based on performance metrics for a plurality of different stains also denoted PM_1_5_1, PM_1_5_2, . . . , such as from 3 to 20 different stains of food thickeners and stabilizers.

A first performance metric PM_1_6 may be indicative of stain removal capability for a sixth stain type, such as other foods and non-foods. Thus, the first performance metric PM_1_6 may be based on performance metrics for a plurality of different stains also denoted PM_1_5_1, PM_1_5_2, . . . , such as from 3 to 20 different stains of other foods and non-food.

One or more first performance metrics, such as PM_1_1, PM_1_2, PM_1_3, and PM_1_4 may indicate stain removal performances for a group of stains. One or more first performance metrics, such as PM_1_1_1-19, PM_1_2_1-9, PM_1_3_1-3, PM_1_4_1-5, PM_1_5_1-3, PM_1_6_1-12 may indicate stain removal performances for individual stains.

In one or more example methods, determining the first performance of the first detergent composition is based on ingredient identifiers and amounts of the ingredient data.

In one or more example methods, determining a first performance is based on the third ingredient data, such as one or more third ingredient identifiers and/or one or more third amounts of the third ingredient data.

In one or more example methods, determining a performance, such as the first performance, of the first detergent composition is based on one or more of the one or more wash conditions. For example, determining a first performance of the first detergent composition may be based on the first wash condition and/or the second wash condition, optionally wherein the first wash condition is indicative of a geographical location and/or the second wash condition is indicative of a wash temperature.

Thus, in one or more example methods, the first wash condition is indicative of a geographical location, and the one or more wash conditions comprises a second wash condition indicative of a wash temperature, and determining a first performance of the first detergent composition is based on the first wash condition and/or the second wash condition.

In one or more exemplary methods/systems/electronic devices, determining a first performance may comprise applying a first performance function or first performance model of stain removal performance. The first performance function or first performance model may take the (first) detergent composition as input and provide one or more first performance metrics indicative of different first performances of the first detergent composition. In one or more exemplary methods/systems/electronic devices, the output of the first performance function or first performance model comprises one or more of a first performance metric PM_1_1 indicative of a stain removal performance on a first type of stains, e.g. AISE, a first performance metric PM_1_2 indicative of a stain removal performance on a second type of stains, e.g. proteins, a first performance metric PM_1_3 indicative of a stain removal performance on a third type of stains, e.g. starches, a first performance metric PM_1_4 indicative of a stain removal performance on a fourth type of stains, e.g. natural fats and oils, a first performance metric PM_1_5 indicative of a stain removal performance on a fifth type of stains, e.g. food thickeners and stabilizers, and a first performance metric PM_1_6 indicative of a stain removal performance on a sixth type of stains, e.g. other foods and non-foods.

The first performance model may be a look-up table mapping the first detergent composition to first performance metrics, for example as defined by one or more performance criteria based on the first detergent composition, such as based on one or more of ingredient identifiers, ingredient type identifiers, and amounts of the first detergent compositions.

The first performance model may be a neural network, e.g. a deep neural network. The neural network may comprise at least 5 hidden layers, such as in the range from 10 to 100 hidden layers. One or more hidden layers, such as a first layer after the input layer may comprise at least 5 nodes, such as at least 20 nodes.

The first performance model may be a neural network with an input layer, one or more hidden layers, such as a plurality of hidden layers, and an output layer.

The input to the first performance model may comprise a stain identifier/stain type identifier. The input to the first performance model may comprise the detergent composition. The detergent composition may be a vector where each vector element in the vector corresponds to an ingredient. The value of a vector element may indicate the amount of the ingredient. The amount of the ingredient may be normalized and/or given in grams/liter.

In one or more exemplary methods/systems/electronic devices, determining a first performance may comprise calling the first performance model at least 10 times, such as at least 25 times, for each stain/stain type, such as for each stain type of a stain set, e.g. AISE stain set, STIWA stain set, JB stain set, JBS stain set, or CHIPS stain set. The stain removal performance of each stain is optionally based on the least 10 outputs, such as the at least 25 outputs.

In one or more exemplary methods/systems/electronic devices, determining a first performance may comprise calling the first performance model with at least 5 different stains or stain types, such as with at least 10 different stains or stain types, such as with at least 20 different stains or stain types, or such as with at least 30 different stains or stain types.

The neural network may comprise a first hidden layer after the input layer. The first hidden layer may comprise at least 5 nodes, such as at least 20 nodes. In one or more exemplary neural networks, the first hidden layer comprises in the range from 100 to 1,000 nodes, such as in the range from 200 to 500 nodes, e.g. about 300 nodes. In one or more exemplary neural networks, the neural network comprises a second hidden layer after the first hidden layer. The second hidden layer optionally comprises in the range from 100 to 1,000 nodes, such as in the range from 200 to 500 nodes, e.g. about 300 nodes. In one or more exemplary neural networks, the neural network has less than 10 hidden layers, such as less than 5 hidden layers.

The output/output layer of the neural network may comprise one or more output variables, such as at least 5 output variables. In one or more exemplary neural networks, the number of output variables is in the range from 6 to 15.

In one or more exemplary methods/systems/electronic devices, determining a first performance may comprise selecting the first performance model from a set of different performance models, e.g. based on a wash condition, such as the first wash condition e.g. indicative of region or geographical location.

The first performance model may be a Random forest model. An example of a suitable Random forest model is a catboost model.

In one or more exemplary methods/systems/electronic devices, determining a first performance may comprise selecting performance metric values from a set of predetermined values, e.g. from a set of predetermined values comprising at least 5 values, at least 10 values, such as at least 50 values.

In one or more exemplary methods/systems/electronic devices, determining a second performance may comprise applying a second performance function or second performance model, e.g. of cost performance. The second performance function or second performance model may take the (first) detergent composition as input and provide one or more second performance metrics indicative of different second performances of the first detergent composition. In one or more exemplary methods/systems/electronic devices, the output of the second performance function or second performance model comprises one or more of a second performance metric PM_2, e.g. indicative of total costs for the first detergent compositions, a second performance metric PM_2_1, e.g. indicative of a cost for first ingredient(s) of a first type, a second performance metric PM_2_2, e.g. indicative of a cost for second ingredient(s) of a second type, a second performance metric PM_2_3, e.g. indicative of a cost for third ingredient(s) of a third type.

The second performance function may take the costs and/or the amounts of the first detergent composition as input and provide the second performance metrics as output.

The method optionally comprises outputting, such as displaying, one or more performance representations, such as a plurality of performance representations and/or at least three performance representations, of performances. In the method displaying one or more performance representations may comprise displaying a first performance representation and/or a second performance representation in a user interface, such as a third user interface, on a display.

For example, the method optionally comprises outputting a first performance representation of the first performance. The first performance representation may be based on one or more first performance metrics of the first detergent composition. The first performance representation may comprise a first primary performance representation based on/indicative of one or more of first performance metrics PM_1_1, PM_1_2, PM_1_3, PM_1_4, PM_1_5, and PM_1_6. In other words, the first primary performance representation optionally illustrates/is indicative of stain removal capability for different stain types. The first performance representation may comprise one or more first secondary performance representations each based on first performance metrics for different stains of the same type. For example, a first secondary performance representation may be based on/indicative of one or more of first performance metrics PM_1_1_1, PM_1_1_2, . . . of stains of a first type, e.g. stains according to AISE or other standard laundry test protocol, such as STIWA, JB, JBS, CHIPS. For example, a first secondary performance representation may be based on/indicative of one or more of first performance metrics PM_1_2_1, PM_1_2_2, . . . of stains of a second type, e.g. protein stains. In one or more exemplary methods, the first performance representation may comprise performance representations for a plurality of standard stain sets. For example, the first performance representation may comprise performance representations for AISE stain set and STIWA stain set, e.g. in regions where more than one stain set standard is applicable.

In one or more example methods, the method comprises determining a second performance of the first detergent composition. Determining a second performance P_2 may comprise determining one or more, such as a plurality of, second performance metrics also denoted PM_2_s, where s=1, . . . , S, S being the number of second performance metrics of the first detergent composition. In other words, the second performance may comprise one or more second performance metrics of the first detergent composition. The second performance, e.g. second performance metric(s), may be based on ingredients and/or ingredient amounts of the first detergent composition. The second performance of the first detergent composition may be based on one or more wash conditions, such as the first wash condition and/or the second wash condition, optionally wherein the first wash condition is indicative of a geographical location and/or the second wash condition is indicative of a wash temperature.

The second performance may be a cost performance. In other words, second performance metric(s) of the second performance may be indicative of cost for the first detergent composition. For example, a second performance metric PM_2_1 may be indicative of costs for a first ingredient type, such as surfactants. Another second performance metric PM_2_2 may be indicative of costs for a second ingredient type, such as enzymes. A second performance metric PM_2_3 may be indicative of costs for a third ingredient type, such as others. A second performance metric PM_2 may be indicative of the total costs for the first detergent composition.

In one or more example methods, the method comprises outputting a second performance representation of the second performance. The second performance representation may be based on one or more second performance metrics of the first detergent composition.

In one or more example methods, the method comprises determining a third performance of the first detergent composition. Determining a third performance may comprise determining one or more, such as a plurality of, third performance metrics also denoted PM_3_t, where t=1, . . . , T, T being the number of third performance metrics of the first detergent composition. In other words, the third performance may comprise one or more third performance metrics of the first detergent composition. The third performance, e.g. third performance metric(s), may be based on ingredients and/or ingredient amounts of the first detergent composition. The third performance of the first detergent composition may be based on one or more wash conditions, such as the first wash condition and/or the second wash condition, optionally wherein the first wash condition is indicative of a geographical location and/or the second wash condition is indicative of a wash temperature.

The third performance may be a sustainability performance. In other words, third performance metric(s) of the third performance may be indicative of sustainability of the first detergent composition.

In one or more exemplary methods/systems/electronic devices, determining a third performance may comprise applying a third performance function or third performance model, e.g. of sustainability performance. The third performance function or third performance model may take the (first) detergent composition and/or wash condition(s) as input and provide one or more third performance metrics indicative of different third performances of the first detergent composition. In one or more exemplary methods/systems/electronic devices, the output of the third performance function or third performance model comprises one or more of a third performance metric PM_3_1, e.g. indicative of a primary sustainability performance, such as CO2 emission, for the first detergent composition, a third performance metric PM_3_2, e.g. indicative of a secondary sustainability performance, such as chemical consumption, for the first detergent composition, a third performance metric PM_3_3, e.g. indicative of a tertiary sustainability performance, such as Critical Dilution Volume (CDV), for the first detergent composition.

The third performance function may take the amounts of the first detergent composition and one or more wash conditions indicative of user pattern or detergent consumption, such as a geographical location, wash pattern, user types, etc. as input and provide the third performance metric(s) as output.

In one or more example methods, the method comprises outputting a third performance representation of the third performance. The third performance representation may be based on one or more third performance metrics of the first detergent composition.

In one or more example methods, the method comprises determining a fourth performance of the first detergent composition. Determining a fourth performance may comprise determining one or more, such as a plurality of, fourth performance metrics also denoted PM_4_1, PM_4_2, PM_4_3, etc., e.g. wherein the number of fourth performance metrics of the first detergent composition is in the range from 1 to 10. In other words, the fourth performance may comprise one or more fourth performance metrics of the first detergent composition. The fourth performance, e.g. fourth performance metric(s), may be based on ingredients and/or ingredient amounts of the first detergent composition. The fourth performance of the first detergent composition may be based on one or more wash conditions, such as the first wash condition and/or the second wash condition, optionally wherein the first wash condition is indicative of a geographical location and/or the second wash condition is indicative of a wash temperature.

The fourth performance may be an Ecolabel performance. In other words, fourth performance metric(s) of the fourth performance may be indicative of whether the first detergent composition satisfies ecolabel criteria. The ecolabel performance may be a performance of a standard ecolabel e.g. as recognized by the EU.

In one or more exemplary methods/systems/electronic devices, determining a fourth performance may comprise applying a fourth performance function or fourth performance model, e.g. of an Ecolabel performance. The fourth performance function or fourth performance model may take the (first) detergent composition and/or wash condition(s) as input and provide one or more fourth performance metrics indicative of different fourth performances of the first detergent composition. In one or more exemplary methods/systems/electronic devices, the output of the fourth performance function or fourth performance model comprises one or more of a fourth performance metric PM_4_1, e.g. indicative of a primary ecolabel performance, such as whether the first detergent composition satisfies a primary ecolabel criterion, a fourth performance metric PM_4_2, e.g. indicative of a secondary ecolabel performance, such as whether the first detergent composition satisfies a secondary ecolabel criterion, and a fourth performance metric PM_4_3, e.g. indicative of a tertiary ecolabel performance, such as whether the first detergent composition satisfies a tertiary ecolabel criterion.

The primary ecolabel criterion may be associated with or indicative of one or more dosage requirements. The secondary ecolabel criterion may be associated with or indicative of one or more toxicity requirements. The tertiary ecolabel criterion may be associated with or indicative of one or more biodegradability requirements.

The fourth performance function may take the ingredients and amounts of the first detergent composition and one or more wash conditions indicative of user pattern or detergent consumption, such as a geographical location, wash pattern, user types, etc. as input and provide the fourth performance metric(s) as output.

In one or more exemplary methods/systems/electronic devices, determining a fourth performance may comprise selecting a fourth primary performance of a first type, e.g. a first ecolabel, in accordance with the first wash condition being indicative of a first geographical location, such as Europe and/or a fourth primary performance of a second type, e.g. a second ecolabel, in accordance with the first wash condition being indicative of a second geographical location, such as US or Asia. Thereby, the fourth performance can be adapted to the respective regions/geographical locations and thus accommodate for different fourth performances used as standard in different regions/geographical locations.

In one or more example methods, the method comprises outputting a fourth performance representation of the fourth performance. The fourth performance representation may be based on one or more fourth performance metrics of the first detergent composition.

In one or more example methods, the method comprises obtaining a primary reference detergent, and obtaining a primary first reference performance associated with the primary reference detergent. The method optionally comprises including a primary first reference performance representation of the primary reference detergent in the first performance representation. Obtaining a primary first reference performance may comprise determining one or more, such as a plurality of, primary first reference performance metrics also denoted RPM_1_1_r, where r=1, . . . , R, R being the number of first performance metrics of the first detergent composition. The primary first reference performance representation may be based on or comprise the primary first reference performance metrics.

The method may comprise obtaining a plurality of reference detergents and obtaining a plurality of first reference performances including primary first reference performance and a secondary first reference performance. The method optionally comprises including the plurality of first reference performances in the first performance representation. Thereby an easy and real-time performance comparison with one or more reference detergents is provided.

In one or more example methods, the method comprises obtaining a primary second reference performance associated with the primary reference detergent. The method optionally comprises including a primary second reference performance representation of the primary reference detergent in the second performance representation. Obtaining a primary second reference performance may comprise determining one or more, such as a plurality of, primary second reference performance metrics also denoted RPM_1_2_s, where s=1, . . . , S, S being the number of second performance metrics of the first detergent composition. The primary second reference performance representation may be based on or comprise the primary second reference performance metrics.

In one or more example methods, the method comprises obtaining an object parameter or a plurality of object parameters. The object parameter may be input via an interface of the electronic device or retrieved from memory. The object parameter may be a default object parameter. The method optionally comprises determining a second detergent composition based on one or more wash conditions, such as the first wash condition and/or the second wash condition, the ingredient data and optionally the object parameter, e.g. in accordance with a detection of user input indicative of detergent composition optimization. The user input indicative of detergent composition optimization may be a user activation of an optimization user interface element of a user interface on a display of the electronic device. Determining the second detergent composition may comprise optimizing the object parameter, e.g. minimizing cost in case the object parameter is cost.

In one or more example methods, the method comprises outputting the second detergent composition, such as displaying, transmitting and/or storing the second detergent composition.

The method optionally comprises determining one or more performances of the second detergent composition; and outputting one or more performance representations of the one or more performances of the second detergent composition as also described with respect to the first detergent composition. In other words, the description of determining performances and outputting performance representations related to the first detergent composition also applies to the second detergent composition. In case, a second detergent composition is determined, the first detergent/first detergent composition may be used as primary reference detergent/detergent composition.

In one or more example methods, the method comprises determining an ingredient representation based on the first detergent composition and optionally displaying the ingredient representation.

In one or more example methods, displaying the ingredient representation comprises displaying, optionally in accordance with a user activation of a first ingredient user interface element, a first ingredient representation indicative of the first ingredients of the first detergent composition, and/or displaying, optionally in accordance with a user activation of a second ingredient user interface element, a second ingredient representation indicative of the second ingredients of the first detergent composition.

The method may comprise receiving a user input, e.g. via interface of the electronic device, indicative of a change in one or more of the first detergent composition and a wash condition, such as the first wash condition and/or the second wash condition. Receiving a user input indicative of a change in the first detergent composition may comprise detecting a user input via the ingredient representation, such as via one or more ingredient sliders and/or arrows of the ingredient representation, optionally followed by detecting a user input indicative of detergent composition performance evaluation.

In one or more example methods, the method comprises determining secondary ingredient data based on the user input indicative of a change in the first detergent composition. The method optionally comprises determining a second detergent composition based on the secondary ingredient data.

In one or more example methods, the method comprises determining a first performance of the second detergent composition and optionally outputting a first performance representation of the first performance of the second detergent composition.

In one or more example methods, the method comprises determining a second performance and/or a third performance of the second detergent composition and outputting a second performance representation of the second performance of the second detergent composition and/or a third performance representation of the third performance of the second detergent composition.

In one or more example methods, the method comprises determining a fourth performance of the second detergent composition and optionally outputting a fourth performance representation of the fourth performance of the second detergent composition.

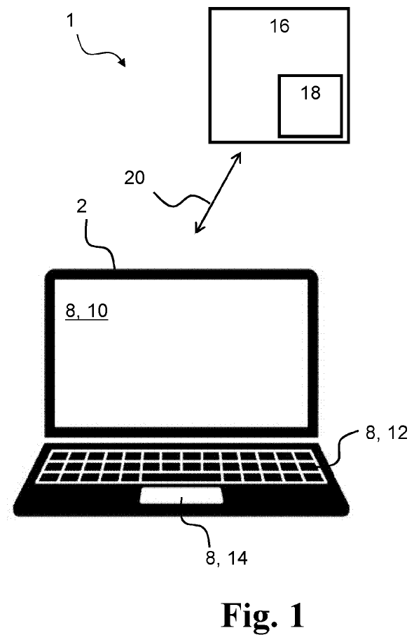

FIG. 1 is a diagram illustrating a system 1 comprising an example electronic device 2 according to this disclosure. The electronic device 2 may be a laptop computer as illustrated, however the electronic device may be a smartphone, tablet computer, a work station, or a stationary computer. The electronic device 2 comprises a processor (not shown but later referred to with ref 4), a memory (not shown but later referred to with ref 6), and an interface 8, the interface 8 comprising a display 10 and optionally a keyboard 12 and/or a pointer device/mouse pad 14. The display 10 may be a touch-sensitive display, e.g. implementing pointer and/or mouse functionality. The processor is configured to obtain one or more wash conditions including a first wash condition and/or a second wash condition via the interface, e.g. by user input via a first user interface on the display 10. To obtain wash conditions may comprise retrieving previously stored wash condition data, default wash condition data, or reference wash condition data, e.g. from the memory and/or from a server device 16/database 18 via wired and/or wireless data connection 20. The processor is configured to obtain ingredient data comprising first ingredient data and second ingredient data via the interface, e.g. by user input via a second user interface on the display 10. To obtain ingredient data may comprise retrieving previously stored ingredient data, default ingredient data, or reference ingredient data, e.g. from the memory and/or from a server device 16/database 18 via wired and/or wireless data connection 20. The first ingredient data is associated with one or more first ingredients including a first primary ingredient and the second ingredient data associated with one or more second ingredients including a second primary ingredient. The processor is configured to determine a first performance including one or more of first performance metrics PM_1_1, PM_1_2, PM_1_3, PM_1_4, PM_1_5, PM_1_6 of the first detergent composition and to output a first performance representation of the first performance via a third user interface on the display. The first performance representation is based on and indicative of the first performance metrics PM_1_1, PM_1_2, PM_1_3, PM_1_4, PM_1_5, PM_1_6.

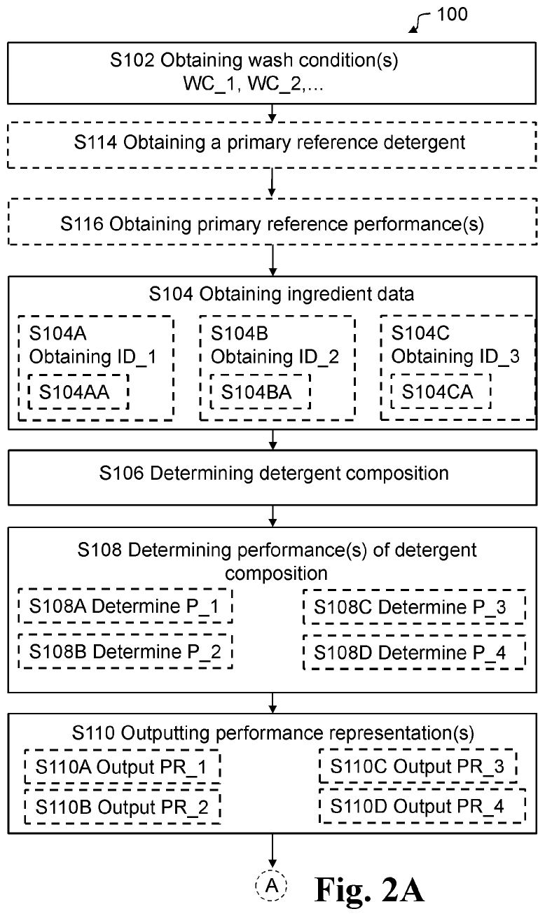

FIGS. 2A and 2B show a flow diagram of an example method of operating a device according to the disclosure. The method 100 is a computer-implemented method for performance evaluation of a detergent composition and/or for optimization of a detergent composition.

The method 102 comprises obtaining S102, e.g. via a first user interface on a display of the electronic device, one or more wash conditions including a first wash condition WC_1 and optionally a second wash condition WC_2. The number of wash conditions may be in the range from 2 to 10. In method 100, the first wash condition WC_1 is a region or country and the second wash condition WC_2 is a wash temperature. The first wash condition is optionally selected from a set of candidate geographic locations, e.g. via a first drop-down list of the first user interface. The second wash condition is optionally selected from a set of candidate wash temperatures, e.g. via a second drop-down list of the first user interface.

The method 100 comprises obtaining S104, e.g. via a second user interface on a display of the electronic device and/or from the memory, ingredient data optionally comprising first ingredient data also denoted ID_1 and/or second ingredient data also denoted ID_2. The ingredient data may comprise third ingredient data also denoted ID_3 and/or fourth ingredient data also denoted ID_4. The first ingredient data ID_1 may comprise costs, e.g. CO_1_1, CO_1_2, CO_1_3, etc. of first ingredients, the second ingredient data ID_2 may comprise costs, e.g. CO_2_1, CO_2_2, CO_2_3, CO_2_4, etc. of second ingredients, and the third ingredient data ID_3 may comprise costs, e.g. CO_3_1, CO_3_2, etc. of third ingredients. Thus, obtaining S104A first ingredient data ID_1 optionally comprises obtaining S104AA one or more costs of respective one or more first ingredients, obtaining S104B second ingredient data ID_2 optionally comprises obtaining S104CA one or more costs of respective one or more second ingredients, and obtaining S104C third ingredient data ID_3 optionally comprises obtaining S104CA one or more costs of respective one or more third ingredients.

Obtaining S104 ingredient data, such as obtaining S104AA, S102BA, S104CA cost(s) of different ingredients may be obtained via value fields of the second interface on display of electronic device. Thereby, a user is allowed to input costs of specific ingredients and to allow determination of a cost performance of the first detergent composition.

The first ingredient data ID_1 are optionally associated with one or more first ingredients including a first primary ingredient also denoted ING_1_1 and/or a first secondary ingredient also denoted ING_1_2. The first ingredients also denoted ING_1_i, where i=1, . . . , N, N being the number of first ingredients, may be of a first ingredient type such as surfactant or enzyme. N may be in the range from 1 to 10, such as from 2 to 5.

The second ingredient data ID_2 are optionally associated with one or more second ingredients including a second primary ingredient also denoted ING_2_1 and/or a second secondary ingredient also denoted ING_2_2. The second ingredients also denoted ING_2j, where j=1, . . . , M, M being the number of second ingredients, may be of a second ingredient type such as surfactant or enzyme. M may be in the range from 1 to 10, such as from 2 to 5.

The third ingredient data ID_3 are optionally associated with one or more third ingredients including a third primary ingredient also denoted ING_3_1 and/or a third secondary ingredient also denoted ING_3_2. The third ingredients also denoted ING_3_k, where k=1, . . . , K, K being the number of third ingredients. K may be in the range from 1 to 10, such as from 2 to 5.

Obtaining ingredient data ID_1, ID_2, ID_3 may comprise retrieving at least a part of the ingredient data ID_1, ID_2, ID_3 from a memory e.g. based on default ingredient data and/or based on a user selecting/indicating previously stored ingredient data.

The method 100 comprises determining S106 a first detergent composition, e.g. based on the ingredient data and/or the one or more wash conditions, such as based one or more, e.g. all, of first ingredient data ID_1, second ingredient data ID_2, and third ingredient data ID_3. In one or more exemplary methods/electronic devices, the first detergent composition is based on fourth ingredient data ID_4. Determining the first detergent composition may comprise including ingredients, such as ingredient identifiers, costs, and amounts of the ingredient data in the first detergent composition.

A detergent composition, such as the first detergent composition, may comprise name/ingredient identifier, cost and amount of the ingredients in the detergent with the detergent composition.

The method 100 comprises determining S108 one or more performances of the first detergent composition.

The method optionally comprises determining S108A a first performance P_1 optionally being a stain removal performance of the first detergent composition. Determining S108A a first performance P_1 optionally comprises determining one or more, such as a plurality of, first performance metrics also denoted PM_1_r, where r=1, . . . , R, R being the number of first performance metrics of the first detergent composition. R may be in the range from 2 to 100. The first performance, e.g. first performance metric(s), may be based on ingredients and/or ingredient amounts of the first detergent composition. The first performance, e.g. first performance metric(s), may be based on one or more wash conditions, e.g. the first wash condition and/or the second wash condition.

The method 100 optionally comprises determining S108B a second performance P_2 optionally being a cost performance of the first detergent composition. Determining a second performance P_2 may comprise determining one or more, such as a plurality of, second performance metrics also denoted PM_2_s, where s=1, . . . , S, S being the number of second performance metrics of the first detergent composition. S may be in the range from 2 to 50. In other words, the second performance may comprise one or more second performance metrics of the first detergent composition. The second performance, e.g. second performance metric(s), may be based on ingredients, costs and/or ingredient amounts of the first detergent composition. The second performance, e.g. second performance metric(s), may be based on one or more wash conditions, e.g. the first wash condition and/or the second wash condition.

The method 100 optionally comprises determining S108C a third performance P_3 optionally being a sustainability performance of the first detergent composition. Determining a third performance P_3 may comprise determining one or more, such as a plurality of, third performance metrics also denoted PM_3_t, where t=1, . . . , T, T being the number of third performance metrics of the first detergent composition. T may be in the range from 2 to 10. In other words, the third performance may comprise one or more third performance metrics of the first detergent composition. The third performance, e.g. third performance metric(s), may be based on ingredients, costs, and/or ingredient amounts of the first detergent composition. The third performance, e.g. third performance metric(s), may be based on one or more wash conditions, e.g. the first wash condition and/or the second wash condition.

The method 100 optionally comprises determining S108D a fourth performance P_4 optionally being an Ecolabel performance of the first detergent composition. Determining a fourth performance P_4 may comprise determining one or more, such as a plurality of, fourth performance metrics also denoted PM_4_1, PM_4_2, PM_4_3, etc., e.g. wherein the number of fourth performance metrics of the first detergent composition is in the range from 1 to 10 such as from 3 to 5. In other words, the fourth performance P_4 may comprise one or more fourth performance metrics of the first detergent composition. The fourth performance, e.g. fourth performance metric(s), may be based on ingredients, costs and/or ingredient amounts of the first detergent composition and may be indicative of whether the first detergent composition satisfies ecolabel criteria. The fourth performance P_4 of the first detergent composition may be based on one or more wash conditions, such as the first wash condition and/or the second wash condition.

The method 100 comprises outputting S110, such as displaying on display and optionally storing in memory, one or more performance representations including one or more of first performance representation PR_1 of first performance P_1, second performance representation PR_2 of second performance P_2, third performance representation PR_3 of third performance P_3, and fourth performance representation PR_4 of fourth performance P_4. The performance representations PR_1, PR_2, PR_3, PR_4 may be displayed in the same user interface, such as a third user interface.

The method 100 optionally comprises obtaining S114 a primary reference detergent, and obtaining S116 one or more primary reference performances, optionally comprising obtaining S116A a primary first reference performance associated with the primary reference detergent and/or obtaining S116B a primary second reference performance associated with the primary reference detergent. Obtaining S116 one or more primary reference performances optionally comprises obtaining S116C a primary third reference performance associated with the primary reference detergent and/or obtaining S116D a primary fourth reference performance associated with the primary reference detergent. Obtaining S114 a primary reference detergent may be performed via the first interface and/or a reference user interface optionally comprising a first drop down list of preset candidate reference detergent compositions and/or a second drop down list of stored candidate reference detergent compositions. Obtaining S116 one or more primary reference performances may comprise retrieving the one or more primary reference performances from a memory and/or determining the one or more primary reference performances based on ingredient data/primary reference detergent composition of the primary reference detergent.

The method 100 optionally comprises including a primary first reference performance representation of the primary reference detergent in the first performance representation e.g. prior to or as part of outputting S110A the first performance representation.

The method 100 optionally comprises including a primary second reference performance representation of the primary reference detergent in the second performance representation e.g. prior to or as part of outputting S110B the second performance representation.

The method 100 optionally comprises including a primary third reference performance representation of the primary reference detergent in the third performance representation e.g. prior to or as part of outputting S110C the third performance representation.

The method 100 optionally comprises including a primary fourth reference performance representation of the primary reference detergent in the fourth performance representation e.g. prior to or as part of outputting S110D the fourth performance representation.

The method 100 may comprise obtaining S118 an object parameter or a plurality of object parameters, e.g. via a drop-down list in a user interface of the display, such as the third user interface. The object parameter may be input via an interface of the electronic device or retrieved from memory. The object parameter may be a default object parameter, such as cost. A plurality of object parameters may be obtained. Thus, a multi-parameter optimization of the performance of the second detergent composition is provided in turn providing a detergent with improved performance. The method optionally comprises determining S120 a second detergent composition based on one or more wash conditions, such as the first wash condition and/or the second wash condition, the ingredient data or secondary ingredient data, and optionally the object parameter, e.g. in accordance with a detection of user input indicative of detergent composition optimization for example via a user activation of an optimization user interface element of a user interface, such as the third user interface, on the display of the electronic device. Determining the second detergent composition may comprise optimizing the object parameter, e.g. minimizing cost in case the object parameter is cost. The method 100 optionally comprises S122 outputting the second detergent composition, such as displaying, transmitting and/or storing the second detergent composition. Outputting S122 the second detergent composition may comprise determining and displaying S122A an ingredient representation based on the second detergent composition via the third user interface of the display and/or determining S122B performance(s) of the second detergent composition followed by outputting S122C performance representation(s) of the performance(s) of the second detergent composition. Outputting S122 the second detergent composition may comprise storing S122D the second detergent composition in memory of the electronic device and/or server device.

The method 100 optionally comprises determining S124 an ingredient representation based on the first detergent composition and/or the ingredient data and optionally displaying S126 the ingredient representation based on the first detergent composition and/or the ingredient data. Displaying S126 the ingredient representation may comprise displaying S126A, optionally in accordance with a user activation of a first ingredient user interface element of the ingredient representation, a first ingredient representation indicative of the first ingredients of the first detergent composition, and/or displaying S126B, optionally in accordance with a user activation of a second ingredient user interface element, a second ingredient representation indicative of the second ingredients of the first detergent composition.

The method 100 optionally comprises receiving S128 user input, e.g. via third user interface on display, i.e. via interface of the electronic device, wherein the user input is indicative of a change in one or more of the first detergent composition and a wash condition, such as the first wash condition and/or the second wash condition. Receiving S128 a user input indicative of a change in one or more of the first detergent composition and a wash condition may comprise detecting S128A a user input indicative of a change in the first detergent composition via the ingredient representation, such as via one or more ingredient sliders and/or arrows of the ingredient representation and/or detecting 128B a user input indicative of a change in a wash condition, such as via a drop down list of the third user interface, optionally followed by detecting S128C a user input indicative of detergent composition performance evaluation for example via a user activation of a performance evaluation user interface element of the third user interface.

In one or more example methods, the method comprises, optionally in accordance with a detection of a user input indicative of detergent composition performance evaluation, determining S130 secondary ingredient data based on the user input indicative of a change in the first detergent composition. The method 100 then proceeds to determining 120 a second detergent composition based on the secondary ingredient data and outputting 122 the second detergent composition as described above including determining a first performance of the second detergent composition and outputting a first performance representation of the first performance of the second detergent composition.

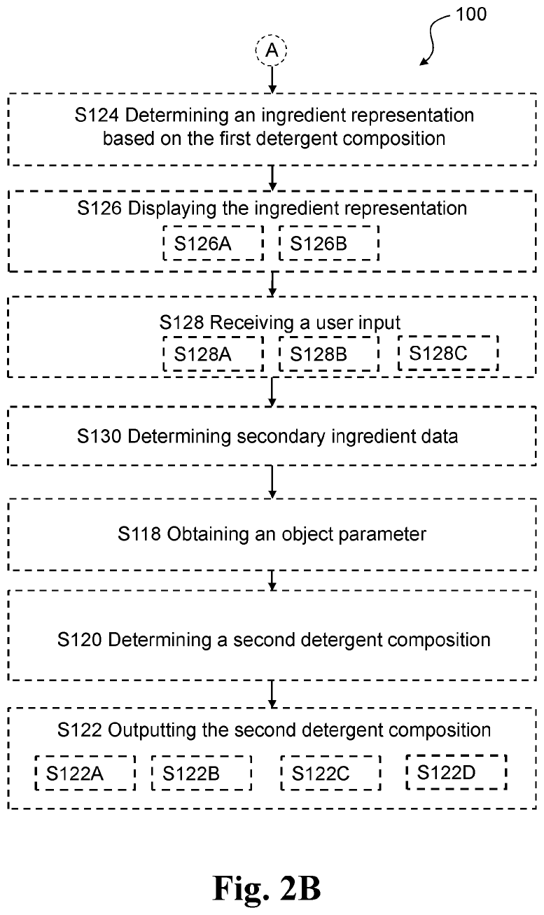

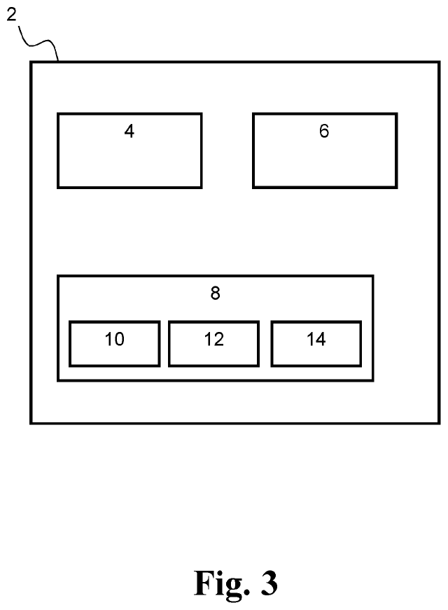

FIG. 3 shows a block diagram of an example electronic device 2 according to the disclosure. The electronic device 2 comprises a processor 4, memory 6, and an interface 8.

Processor 4 is optionally configured to perform any of the operations disclosed in FIGS. 2A and 2B (such as any one or more of S102, S104, S106, S108, S110, S114, S116, S118, S120, S122). The operations of the electronic device 2 may be embodied in the form of executable logic routines (for example, lines of code, software programs, etc.) that are stored on a non-transitory computer readable medium (for example, memory or memory circuitry 6) and are executed by processor or processor circuitry 4).

Furthermore, the operations of the electronic device 2 may be considered a method that the electronic device 2 is configured to carry out. Also, while the described functions and operations may be implemented in software, such functionality may as well be carried out via dedicated hardware or firmware, or some combination of hardware, firmware and/or software.

Memory 6 may be one or more of a buffer, a flash memory, a hard drive, a removable media, a volatile memory, a non-volatile memory, a random access memory (RAM), or other suitable device. In a typical arrangement, memory 6 may include a non-volatile memory for long term data storage and a volatile memory that functions as system memory for processor 4. Memory 6 may exchange data with processor 4 over a data bus. Control lines and an address bus between memory 6 and processor 4 also may be present (not shown in FIG. 3). Memory 6 is considered a non-transitory computer readable medium.

Memory 6 may be configured to store one or more of wash conditions, ingredient data, detergent composition(s), performance(s), and performance representation(s) in a part of the memory 6.

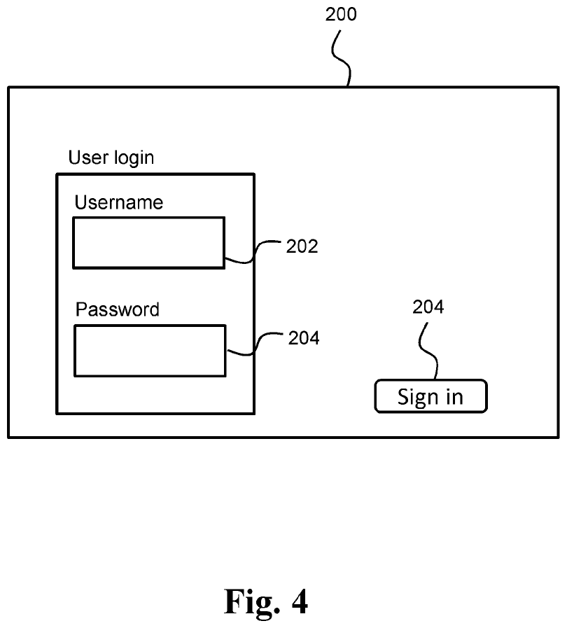

FIG. 4 shows an exemplary initial user interface 200 optionally used in the method/application as described herein. The initial user interface 200 allows to control access to the application and functionality of the application by login via username input field 202 and password field 204 of the initial user interface 200. Upon inputting username and password, a user signs in by pressing or activating the sign-in user interface element 206.

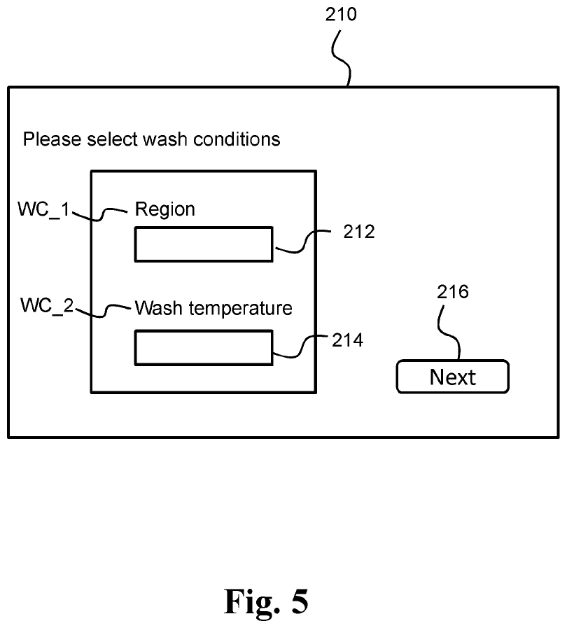

FIG. 5 shows an exemplary first user interface 210 optionally used in the method/application as described herein for obtaining one or more wash conditions, e.g. in S102 of FIG. 2A. The user inputs wash condition(s) including a first wash condition WC_1 being a region via first user input field 212 and a second wash condition WC_2 being a wash temperature via second user input field 214. The first user input field 212 and the second user input field 214 are drop-down lists, the first drop-down list of the first user input field 212 comprising a number of candidate regions/geographical locations and the second drop-down list of the second user input field 214 comprising a number of candidate wash temperatures, such a plurality of wash temperatures of 20 degrees, 30 degrees, 40 degrees, 60 degrees, and 90 degrees. When the user has selected first and second wash conditions, the application/method proceeds, when the user presses or activates next user interface element 216.

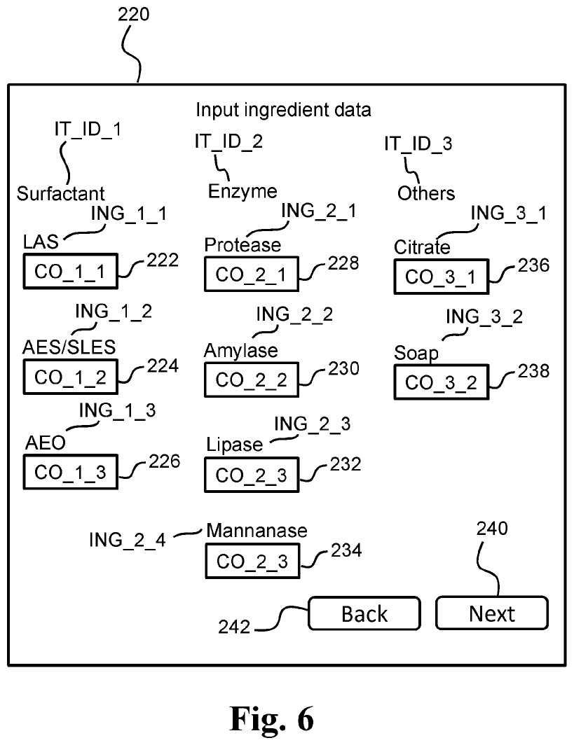

FIG. 6 shows an exemplary second user interface 220 optionally used in the method/application as described herein for obtaining ingredient data, e.g. in S104 of FIG. 2A. The ingredients shown in the second user interface may be default ingredients and/or indicated by the user, e.g. in a reference input user interface used in S116 of FIG. 2A.

The user inputs ingredient data, such as cost(s) CO_1_1, CO_1_2, and CO_1_3 of first ingredient data ID_1 for three first ingredients ING_1_1, ING_1_2, ING_1_3 of a first type as identified by first ingredient type identifier IT_ID_1 being surfactant via cost input fields 222, 224, 226. The user inputs ingredient data, such as cost(s) CO_2_1, CO_2_2, CO_2_3, and CO_2_4 of second ingredient data ID_2 for four second ingredients ING_2_1, ING_2_2, ING_2_3, ING_2_4 of a second type as identified by second ingredient type identifier IT_ID_2 being enzyme via cost input fields 228, 230, 232, 234. The user inputs ingredient data, such as cost(s) CO_3_1 and CO_3_2 of third ingredient data ID_3 for two third ingredients ING_3_1, ING_3_2 of a third type as identified by third ingredient type identifier IT_ID_3 being others via cost input fields 236, 238.

When the user has input ingredient data via some or all input fields 222, 224, 226, 228, 230, 232, 234, 236, 238, the application/method proceeds, when the user presses or activates next user interface element 240 or returns to previous user interface 210 when the user presses or activates back user interface element 242.

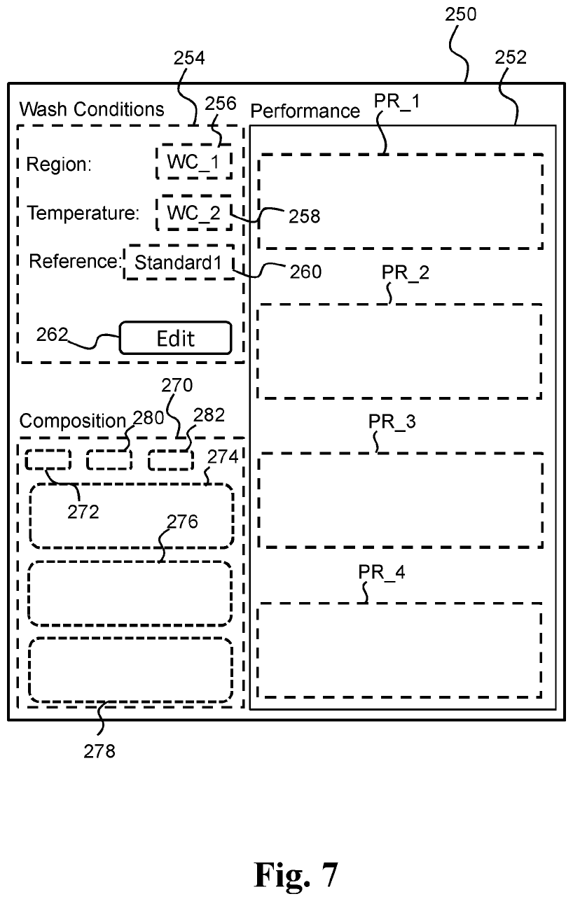

FIG. 7 shows an exemplary third user interface 250 optionally used in the method/application as described herein for outputting performance representations, e.g. in S110 of FIG. 2A. The third user interface 250 comprises one or more performance representations including one or more of a first performance representation PR_1 indicative of the first performance P_1 of the first detergent composition, a second performance representation PR_2 indicative of the second performance of the first detergent composition, a third performance representation PR_3 indicative of the third performance of the first detergent composition, and a fourth performance representation PR_4 of the fourth performance of the first detergent composition. The user interface may be divided into a plurality of regions including a performance region 252 including performance representations PR_1, PR_2, PR_3, and PR_4. The performance region 252 may comprise a performance scrolling function for up-down and/or left/right scrolling of the performance representations.

The third user interface 250 optionally comprises a wash condition representation 254 indicative of wash conditions, e.g. obtained in S102, including a first output field 256 indicative of the first wash condition WC_1 as selected earlier and a second output field 258 indicative of the second wash condition WC_2 as selected earlier. Optionally, the wash condition representation 254 comprises a reference output field 260 indicative of a reference detergent, such as a primary reference detergent named Standard1, optionally used in the method/application as described, e.g. in relation to S110, S114 and S116 of FIG. 2A.

Optionally, the wash condition representation 254 comprises an edit user interface element 262. When the user presses or activates the edit user interface element 262, i.e. in accordance with detecting a user input indicative of wash condition and/or reference edit, one/or more of the output fields 254, 256, 258 switches to respective drop-down lists comprising a set of candidate wash conditions for wash conditions and a set of candidate reference detergents for the reference detergent. At the same time, the edit user interface element 262 changes to an update user interface element, and when the user presses or activates the update user interface element, the method/application proceeds to determining updated performance(s) of the first detergent composition based on the updated wash condition(s); and outputting updated performance representation(s) PR_1, PR_2, PR_3, PR_4 of the first performance, e.g. as described herein in relation to S108 and S110 of FIG. 2A.

The third user interface 250 optionally comprises an ingredient representation 270 indicative of the first detergent composition, optionally used in the method/application as described herein for determining S124 an ingredient representation based on the first detergent composition and/or the ingredient data and optionally displaying S126 the ingredient representation based on the first detergent composition and/or the ingredient data, e.g. as described in relation to FIG. 2B. The ingredient representation 270 optionally comprises representations of the first ingredients, e.g. in accordance with user activation of first ingredient user interface element 272 as shown with first ingredient representations 274, 276, 278 of first primary ingredient ING_1_1, first secondary ingredient ING_1_2, and first tertiary ingredient ING_1_3, respectively. The ingredient representation 270 optionally comprises representations of the second ingredients, e.g. in accordance with user activation of second ingredient user interface element 280. The ingredient representation 270 optionally comprises representations of the third ingredients, e.g. in accordance with user activation of third ingredient user interface element 282. The representations 274, 276, 278 of the first ingredients may be displayed as default.

The wash condition representation 254 and the ingredient representation 270 may be arranged in a detergent region of the plurality of regions. The detergent region may comprise a detergent scrolling function for up-down and/or left/right scrolling of the wash condition representation 254 and/or the ingredient representation 270. The detergent region may be arranged side-by-side with the performance region. Thereby, efficient display and use of the display is provided.

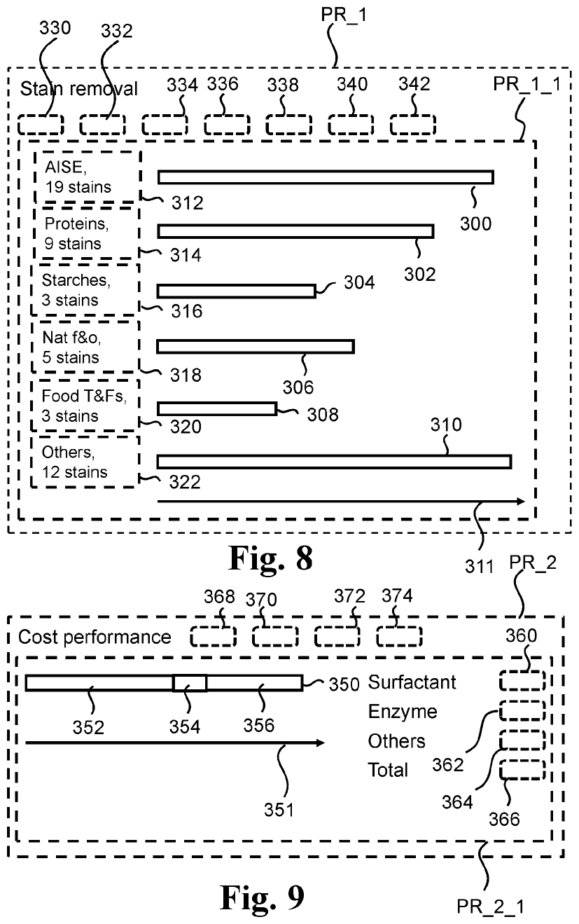

FIG. 8 shows an exemplary first performance representation of a first performance being stain removal. The first performance representation PR_1 comprises a first primary performance representation PR_1_1 being a vertical bar chart that is based on first performance metrics of the first detergent composition. The first primary performance representation PR_1_1 is based on first performance metrics PM_1_1, PM_1_2, PM_1_3, PM_1_4, PM_1_5, and PM_1_6 for respective stain types, where each bar 300, 302, 304, 306, 308, 310 are indicative of a value of the first performance metrics PM_1_1, PM_1_2, PM_1_3, PM_1_4, PM_1_5, and PM_1_6, respectively on a scale indicated with arrow 311. Text fields 312, 314, 316, 318, 320 indicate respective groups/types of the performance metrics. For example, the text field 312 with the text AISE and 19 stains indicates that the bar 300 is indicative of a first performance metric PM_1_1 for stains of the AISE type and optionally based on the performance metrics PM_1_1_1-19 for the 19 individual stains of the AISE type. For example, the text field 314 with the text Proteins and 9 stains indicates that the bar 302 is indicative of a first performance metric PM_1_2 for stains of the protein type and optionally based on the performance metrics PM_1_2_1-9 for the 9 individual stains of the protein type. In other words, the first primary performance representation PR_1_1 optionally illustrates/is indicative of stain removal capability for different stain types. The first performance representation may comprise one or more first secondary performance representations each based on first performance metrics for different stains of the same type. For example, a first secondary performance representation may be based on/indicative of one or more of first performance metrics PM_1_1_1, PM_1_1_2, . . . of stains of a first type, e.g. stains according to AISE. For example, a first secondary performance representation may be based on/indicative of one or more of first performance metrics PM_1_2_1, PM_1_2_2, . . . of stains of a second type, e.g. protein stains. The first performance representation PR_1 of FIG. 8 optionally comprises a plurality of representation selection user interface elements or a drop down user interface element allowing a user to switch between the first primary performance representation as selected by representation selection user interface element 330 or first element of a drop down list and one or more first secondary performance representation as selected by representation selection user interface elements 332, 334, 336, 338, 340, 342 or further elements of a drop down list. In one or more exemplary methods/electronic devices, the number of first secondary performance representations is two or more. In other words, the first performance representation may be indicative of performance on a plurality of stain types. It is to be noted that other chart types may be used.

FIG. 9 shows an exemplary second performance representation of a second performance being cost performance. The second performance representation PR_2 comprises second primary performance representation PR_2_1 comprising a bar chart with a bar 350 indicating the total costs, i.e. second performance metric PM_2, of the first detergent composition on a scale indicated by arrow 351. The bar 350 comprises a first part 352 indicative of the costs of the first ingredients of the first type, i.e. second performance metric PM_2_1, a second part 354 indicative of the costs of the second ingredients of the second type, i.e. second performance metric PM_2_2 and optionally a third part 356 indicative of the costs of the third ingredients of the third type, i.e. second performance metric PM_2_3. In other words, the second performance representation is based on one or more second performance metrics of the second performance. Optionally, the second primary performance representation PR_2_1 comprises text fields indicative of the content of second performance metrics and output fields 260, 362, 364, 366 indicating values of second performance metrics, PM_2_1, PM_2_2, PM_2_3, and PM_2, respectively. The parts 352, 354, 356 may be color-coded with different colors.

The second performance representation PR_2 of FIG. 9 optionally comprises a plurality of representation selection user interface elements or a drop down user interface element allowing a user to switch between the second primary performance representation as selected by representation selection user interface element 368 or first element of a drop down list and one or more first secondary performance representation as selected by representation selection user interface elements 370, 372, 374 or further elements of a drop down list. In one or more exemplary methods/electronic devices, the number of second secondary performance representations is two or more. In other words, the second performance representation may be indicative of performance on a plurality of ingredient types. It is to be noted that other chart types may be used.

FIG. 10 shows an exemplary third performance representation of a third performance being sustainability performance. The third performance representation PR_3 comprises one or more bars 400, 402, 404 indicating third performance metrics PM_3_1, PM_3_2, and PM_3_3, respectively on different scales indicated by arrows 406, 408, 410. The third performance metric PM_3_1 is indicative of a primary sustainability performance as indicated with text field 412, such as CO2 emission, for the first detergent composition. The third performance metric PM_3_2 is indicative of a secondary sustainability performance as indicated with text field 414, such as chemical consumption, for the first detergent composition. The third performance metric PM_3_3 is indicative of a tertiary sustainability performance as indicated with text field 416, such as Critical Dilution Volume (CDV), for the first detergent composition. It is to be noted that other chart types may be used.

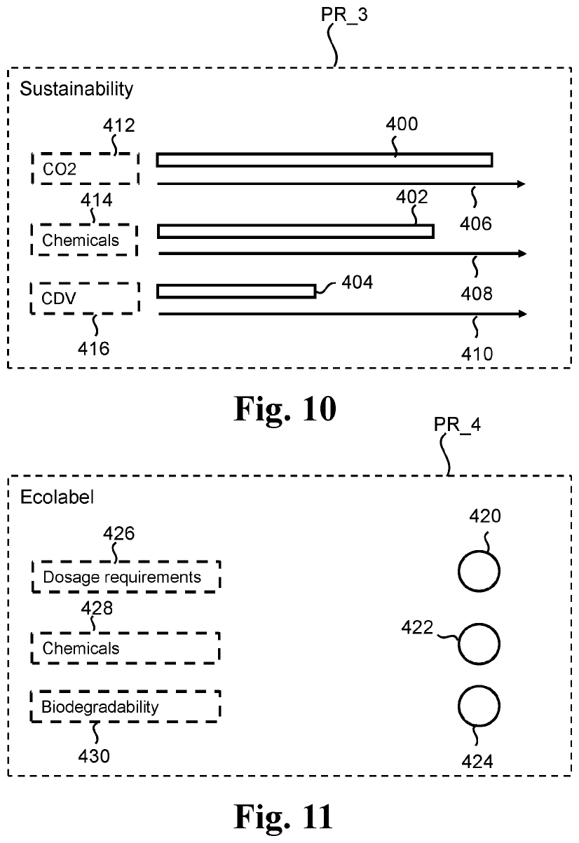

FIG. 11 shows an exemplary fourth performance representation of a fourth performance being ecolabel performance. The fourth performance representation PR_4 comprises one or more graphical user interfaces 420, 422, 424 indicating fourth performance metrics PM_4_1, PM_4_2, and PM_4_3, respectively. The fourth performance metric PM_4_1 is indicative of a indicative of a primary ecolabel performance, such as whether the first detergent composition satisfies a primary ecolabel criterion, the fourth performance metric PM_4_2 is indicative of a secondary ecolabel performance, such as whether the first detergent composition satisfies a secondary ecolabel criterion, and the fourth performance metric PM_4_3 is indicative of a tertiary ecolabel performance, such as whether the first detergent composition satisfies a tertiary ecolabel criterion. The fourth performance metric PM_4_1 and the primary ecolabel criterion is indicative of one or more dosage requirements as indicated with text field 426. The fourth performance metric PM_4_2 and the secondary ecolabel criterion is indicative of one or more toxicity requirements as indicated with text field 428. The fourth performance metric PM_4_3 and the tertiary ecolabel criterion is indicative of one or more biodegradability requirements as indicated with text field 430. The graphical user interfaces 420, 422, 424 may be encoded with one or more of a first color, first size and first shape if respective ecolabel criterion is satisfied and with one or more of a second color, second size and second shape if respective ecolabel criterion is not satisfied. In other words, a first graphical user interface type may be displayed if a respective ecolabel criterion is satisfied and a second graphical user interface type may be used if a respective ecolabel criterion is not satisfied.

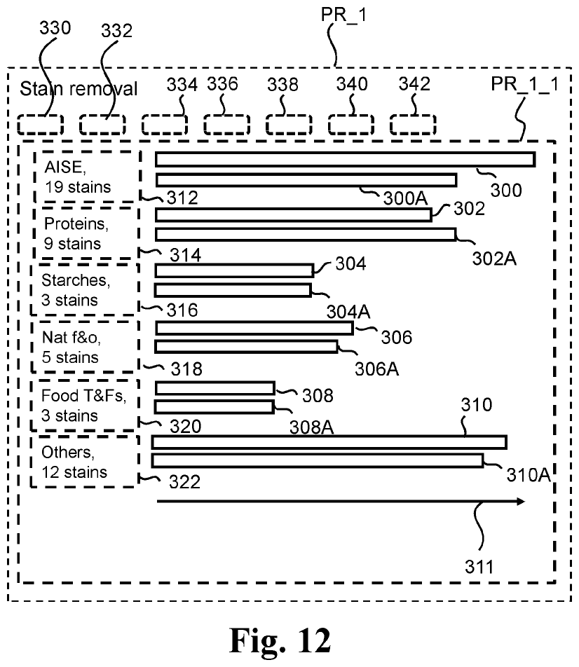

FIG. 12 shows an exemplary first performance representation of a first performance being stain removal. The first performance representation PR_1 is based on a primary reference detergent composition with associated primary reference performance. Reference performance representations are included in the first performance representation to allow a user to easily and fast see how well the first detergent composition performs compared to a reference detergent composition.

The first primary performance representation PR_1_1 is based on first reference performance metrics RPM_1_1, RPM_1_2, RPM_1_3, RPM_1_4, RPM_1_5, and RPM_1_6 for the respective stain types, where each bar 300A, 302A, 304A, 306A, 308A, 310A are indicative of a value of the first reference performance metrics RPM_1_1, RPM_1_2, RPM_1_3, RPM_1_4, RPM_1_5, and RPM_1_6, respectively on the scale indicated with arrow 311.

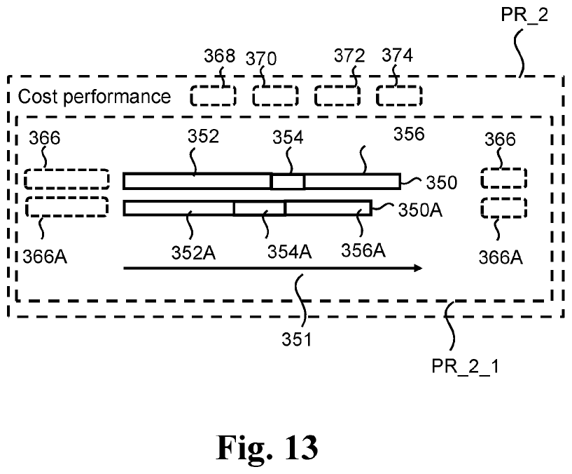

FIG. 13 shows an exemplary second performance representation including associated primary reference performance of primary reference detergent composition. The second performance representation PR_2 comprises second primary reference performance representation comprising a bar 350A indicating the total costs, i.e. second reference performance metric RPM_2, of the primary reference detergent composition on the scale indicated by arrow 351. The bar 350A comprises a first part 352A indicative of the costs of the first ingredients of the first type, i.e. second reference performance metric RPM_2_1, a second part 354A indicative of the costs of the second ingredients of the second type, i.e. second reference performance metric RPM_2_2 and optionally a third part 356A indicative of the costs of the third ingredients of the third type, i.e. second reference performance metric RPM_2_3. As seen, the total cost for the first detergent composition is higher than the total cost for the primary reference detergent composition. The parts 352A, 354A, 356A may be color-coded with different colors. Optionally, the second primary performance representation PR_2_1 comprises output fields 366, 366A, indicating values of second performance metrics PM_2 and RPM_2 being total costs related to first detergent composition and primary reference detergent composition, respectively. Text fields 368 and/or 368A indicate respective names, such as file names, associated with the first detergent composition and/or the primary reference detergent composition, respectively.

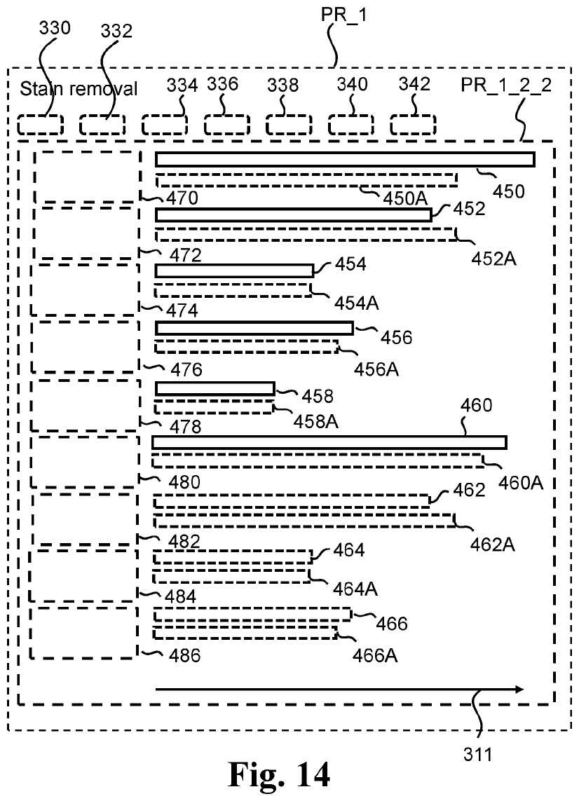

FIG. 14 shows an exemplary first performance representation PR_1 of a first performance being stain removal. The first performance representation PR_1 comprises a first secondary performance representation PR_1_2_2 being a vertical bar chart that is based on first performance metrics of the first detergent composition and optionally first primary reference performance metrics of a primary reference detergent composition. The first secondary performance representation PR_1_2_2 is based on the nine first performance metrics PM_1_2_1, P_1_2_2, . . . , PM_1_2_9, indicative of first secondary performances for stains of the second type being protein stains. Each bar 450, 452, 452, 454, 456, 458, 460, 462, 464, 466 are indicative of a value of the first performance metrics PM_1_2_1, PM_1_2_2, PM_1_2_3, PM_1_2_4, PM_1_2_5, PM_1_2_6, PM_1_2_7, PM_1_2_8, PM_1_2_9, respectively on a scale indicated with arrow 311. The scale 311 may be a REM scale.

The first secondary performance representation PR_1_2_2 is displayed in accordance with the user activating representation selection user interface element 334 or a first element of a drop down list.

Text fields 470, 472, 474, 476, 478, 480, 482, 484, 486 indicate respective proteins of the second type. For example, the text field 470 may have the text Blood, sheep blood indicative of the first performance metric PM_1_2_1 being indicative of the first detergent composition stain removal performance on blood, sheep blood. For example, the text field 472 may have the text Blood/milk/ink indicative of the first performance metric PM_1_2_2 being indicative of the first detergent composition stain removal performance on Blood/milk/ink. For example, the text field 474 may have the text Chocolate milk, pure indicative of the first performance metric PM_1_2_3 being indicative of the first detergent composition stain removal performance on Chocolate milk, pure. For example, the text field 476 may have the text Egg/carbon black indicative of the first performance metric PM_1_2_4 being indicative of the first detergent composition stain removal performance on Egg/carbon black. For example, the text field 478 may have the text Grass (CFT) indicative of the first performance metric PM_1_2_5 being indicative of the first detergent composition stain removal performance on Grass (CFT). For example, the text field 480 may have the text Chocolate milk indicative of the first performance metric PM_1_2_6 being indicative of the first detergent composition stain removal performance on Chocolate milk. For example, the text field 482 may have the text Grass (EPMA) indicative of the first performance metric PM_1_2_7 being indicative of the first detergent composition stain removal performance on Grass (EPMA). For example, the text field 484 may have the text Grass, extract indicative of the first performance metric PM_1_2_8 being indicative of the first detergent composition stain removal performance on Grass, extract. For example, the text field 486 may have the text Pigment/oil/milk indicative of the first performance metric PM_1_2_9 being indicative of the first detergent composition stain removal performance on Pigment/oil/milk.

The first secondary performance representation PR_1_2_2 is optionally based on first reference performance metrics RPM_1_2_1, RPM_1_2_2, . . . , RPM_1_2_9 for the respective stains of the second type, where each bar 450A, 452A, 454A, 456A, 458A, 460A, 462A, 464A, 466A are indicative of a value of the first reference performance metrics RPM_1_2_1, RPM_1_2_2, . . . , RPM_1_2_9, respectively on the scale indicated with arrow 311.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It may be appreciated that FIGS. 1-14 comprises some circuitries or operations which are illustrated with a solid line and some circuitries or operations which are illustrated with a dashed line. Circuitries or operations which are comprised in a solid line are circuitries or operations which are comprised in the broadest example embodiment. Circuitries or operations which are comprised in a dashed line are example embodiments which may be comprised in, or a part of, or are further circuitries or operations which may be taken in addition to circuitries or operations of the solid line example embodiments. It should be appreciated that these operations need not be performed in order presented. Furthermore, it should be appreciated that not all of the operations need to be performed. The example operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the example embodiments may be implemented at least in part by means of both hardware and software, and that several “means”, “units” or “devices” may be represented by the same item of hardware.

The various example methods, devices, nodes and systems described herein are described in the general context of method steps or processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program circuitries may include routines, programs, objects, components, data structures, etc. that perform specified tasks or implement specific abstract data types. Computer-executable instructions, associated data structures, and program circuitries represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly, to be regarded in an illustrative rather than restrictive sense. The claimed disclosure is intended to cover all alternatives, modifications, and equivalents.

### LIST OF REFERENCES

1 detergent composition system
2 electronic device
4 processor
6 memory
8 interface
10 display
12 keyboard
14 pointer device/mouse pad
16 server device
18 database
20 wired and/or wireless data connection
100 method for performance evaluation of detergent composition
S102 obtaining one or more wash conditions
S104 obtaining ingredient data
S104A obtaining first ingredient data
S104AA obtaining costs of first ingredients
S104B obtaining second ingredient data
S104BA obtaining costs of second ingredients
S104C obtaining third ingredient data
S104CA obtaining costs of third ingredients
S106 determining detergent composition based on ingredient data
S108 determining performance(s) of detergent composition
S108A determining first performance
S108B determining second performance
S108C determining third performance
S108D determining fourth performance
S110 outputting performance representations
S110A outputting first performance representation
S110B outputting second performance representation
S110C outputting third performance representation
S110D outputting fourth performance representation
S114 obtaining a primary reference detergent
S116 obtaining one or more primary reference performances of primary reference detergent
S118 obtaining an object parameter
S120 determining a second detergent composition
S122 outputting the second detergent composition
S122A displaying an ingredient representation
S122B determining performance(s) of the second detergent composition
S122C outputting performance representation(s) of the performance(s) of the second detergent composition
S122D storing the second detergent composition in memory of the electronic device and/or server device.
S124 determining ingredient representation based on the first detergent composition and/or the ingredient data
S126 displaying the ingredient representation
S126A displaying a first ingredient representation indicative of the first ingredients of the first detergent composition
S126B displaying a second ingredient representation indicative of the second ingredients of the first detergent composition.
S128 receiving user input
S128A detecting a user input indicative of a change in the first detergent composition
S128B detecting a user input indicative of a change in a wash condition
S128C detecting a user input indicative of detergent composition performance evaluation
S130 determining secondary ingredient data
200 initial user interface
202 username input field
204 password input field
206 sign-in user interface element
210 first user interface
212 first user input field of first user interface
214 second user input field of first user interface
216 next user interface element
220 second user interface
222 cost input field for first primary ingredient
224 cost input field for first secondary ingredient
226 cost input field for first tertiary ingredient
228 cost input field for second primary ingredient
230 cost input field for second secondary ingredient
232 cost input field for second tertiary ingredient
234 cost input field for second quaternary ingredient
236 cost input field for third primary ingredient
238 cost input field for third secondary ingredient
240 next user interface element
242 back user interface element
250 third user interface
252 performance region
254 wash condition representation
256 first output field
258 second output field
260 reference output field
262 edit user interface element
270 ingredient representation
272 first ingredient user interface element
274 first ingredient representation of first primary ingredient
276 first ingredient representation of first secondary ingredient
278 first ingredient representation of first tertiary ingredient
280 second ingredient user interface element
282 third ingredient user interface element
300 bar indicative of PM_1_1
302 bar indicative of PM_1_2
304 bar indicative of PM_1_3
306 bar indicative of PM_1_4
308 bar indicative of PM_1_5
310 bar indicative of PM_1_6
311 scale for first performance metrics
312 text field indicative of type of first stains and/or number of first stains
314 text field indicative of type of second stains and/or number of second stains
316 text field indicative of type of third stains and/or number of third stains
318 text field indicative of type of fourth stains and/or number of fourth stains
320 text field indicative of type of fifth stains and/or number of fifth stains
322 text field indicative of type of sixth stains and/or number of sixth stains
332 representation selection user interface element for first type of stains
334 representation selection user interface element for second type of stains
336 representation selection user interface element for third type of stains
338 representation selection user interface element for fourth type of stains
340 representation selection user interface element for fifth type of stains
342 representation selection user interface element for sixth type of stains
350 bar indicative of total cost and cost for different ingredient types
351 scale for second performance metrics
352 first part indicative of total cost of first ingredients or PM_2_1
354 second part indicative of total cost of second ingredients or PM_2_2
356 third part indicative of total cost of third ingredients or PM_2_3
360 output field indicative of second performance metric PM_2_1
362 output field indicative of second performance metric PM_2_2
364 output field indicative of second performance metric PM_2_3
366 output field indicative of second performance metric PM_2
368 text field with name of first detergent composition
368A text field with name of primary reference detergent composition
400 bar indicative of PM_3_1
402 bar indicative of PM_3_2
404 bar indicative of PM_3_3
406 scale for PM_3_1
408 scale for PM_3_2
410 scale for PM_3_3
412 text field
414 text field
450 bar indicative of PM_1_2_1
452 bar indicative of PM_1_2_2
454 bar indicative of PM_1_2_3
456 bar indicative of PM_1_2_4
458 bar indicative of PM_1_2_5
460 bar indicative of PM_1_2_6
462 bar indicative of PM_1_2_7
464 bar indicative of PM_1_2_8
466 bar indicative of PM_1_2_9
470 text field indicative of first protein of second type of second stain
472 text field indicative of second protein of second type of second stain
474 text field indicative of third protein of second type of second stain
476 text field indicative of fourth protein of second type of second stain
478 text field indicative of fifth protein of second type of second stain
480 text field indicative of sixth protein of second type of second stain
482 text field indicative of seventh protein of second type of second stain
484 text field indicative of eighth protein of second type of second stain
486 text field indicative of ninth protein of second type of second stain
PR_1 first performance representation
PR_2 second performance representation
PR_3 third performance representation
PR_4 four performance representation
WC_1 first wash condition
WC_2 second wash condition

1. A computer-implemented method for performance evaluation of a detergent composition, the method comprising:
obtaining one or more wash conditions including a first wash condition;
obtaining ingredient data comprising first ingredient data and second ingredient data, the first ingredient data associated with one or more first ingredients including a first primary ingredient and the second ingredient data associated with one or more second ingredients including a second primary ingredient;
determining a first detergent composition based on the ingredient data;
determining a first performance of the first detergent composition; and
outputting a first performance representation of the first performance.

2. The method according to claim 1, wherein the method comprises determining a second performance of the first detergent composition and outputting a second performance representation of the second performance.

3. The method according to claim 1, wherein the method comprises determining a third performance of the first detergent composition and outputting a third performance representation of the third performance.

4. The method according to claim 1, wherein the first wash condition is indicative of a geographical location, and the one or more wash conditions comprises a second wash condition indicative of a wash temperature, wherein determining a first performance of the first detergent composition is based on the first wash condition and/or the second wash condition.

5. The method according to claim 1, wherein the first primary ingredient is a surfactant, and the first ingredient data comprises first primary ingredient data of the first primary ingredient, the first primary ingredient data including one or more of identifiers, an amount, a cost, a purity parameter, an impurity parameter, a form parameter, a size parameter, a lower limit, and an upper limit.

6. The method according to claim 1, wherein the second primary ingredient is an enzyme, and the second ingredient data comprises second primary ingredient data of the second primary ingredient, the second primary ingredient data including one or more of identifiers, an amount, a cost, a purity parameter, an impurity parameter, a form parameter, a size parameter, a lower limit, and an upper limit.

7. The method according to according to claim 1, wherein obtaining ingredient data comprises obtaining third ingredient data associated with one or more third ingredients including a third primary ingredient, and wherein determining a first performance is based on the third ingredient data.

8. The method according to claim 1, wherein the method comprises obtaining a primary reference detergent, obtaining a primary first reference performance associated with the primary reference detergent and including a primary first reference performance representation of the primary reference detergent in the first performance representation.

9. The method according to claim 1, wherein the method comprises:
obtaining an object parameter;
determining a second detergent composition based on the first wash condition, the ingredient data and the object parameter; and
outputting the second detergent composition.

10. The method according to claim 1, the method comprising:
determining an ingredient representation based on the first detergent composition; and
displaying the ingredient representation, wherein displaying the ingredient representation comprises displaying, in accordance with a user activation of a first ingredient user interface element, a first ingredient representation indicative of the first ingredients of the first detergent composition, and displaying, in accordance with a user activation of a second ingredient user interface element, a second ingredient representation indicative of the second ingredients of the first detergent composition.

11. The method according to claim 10, the method comprising:
receiving a user input indicative of a change in one or more of the first detergent composition and the first wash condition;
determining secondary ingredient data based on the user input indicative of a change in the first detergent composition;
determining a second detergent composition based on the secondary ingredient data;
determining a first performance of the second detergent composition; and
outputting a first performance representation of the first performance of the second detergent composition.

12. The method according to claim 11, wherein the method comprises determining a second performance and a third performance of the second detergent composition and outputting a second performance representation of the second performance of the second detergent composition and a third performance representation of the third performance of the second detergent composition.

13. The method according to claim 12, wherein the method comprises determining a fourth performance of the second detergent composition and outputting a fourth performance representation of the third performance of the second detergent composition.

14. An electronic device comprising a processor, a memory, and an interface, wherein the processor is configured to:
obtain one or more wash conditions including a first wash condition;
obtain ingredient data comprising first ingredient data and second ingredient data, the first ingredient data associated with one or more first ingredients including a first primary ingredient and the second ingredient data associated with one or more second ingredients including a second primary ingredient;
determine a first performance of the first detergent composition; and
output a first performance representation of the first performance.
